(19) United States
(12) Patent Application Publication
(10) Pub. No.: US 2026/0061187 A1
(43) Pub. Date: Mar. 5, 2026

(54) SYSTEMS AND METHODS FOR IMPLANTABLE MEDICAL DEVICE ANCHORS

(71) Applicant: Advanced Neuromodulation Systems, Inc., Plano, TX (US)

(72) Inventor: Aaron Raines, Dallas, TX (US)

(21) Appl. No.: 19/313,269

(22) Filed: Aug. 28, 2025

Related U.S. Application Data
(60) Provisional application No. 63/688,638, filed on Aug. 29, 2024.

(51) Int. Cl. A61N 1/05

(52) U.S. Cl. CPC A61N 1/057

(57) ABSTRACT

An anchor assembly is provided. The anchor assembly includes a holder defining a passageway therethrough, and a collapsible tube positioned partially within the passageway, the collapsible tube slidable relative to the holder to transition the anchor assembly between i) an unlocked position in which a medical device lead extending through the anchor assembly is slidable relative to the anchor assembly and ii) a locked position in which the medical device lead is fixedly coupled to the anchor assembly.

### CROSS-REFERENCE TO RELATED APPLICATION

This application claims priority to U.S. Provisional Patent Application No. 63/688,638 filed on Aug. 29, 2024, which is incorporated by reference herein in its entirety.

### BACKGROUND OF THE DISCLOSURE

### A. Field of the Disclosure

The present disclosure relates generally to medical device leads, and more particularly, to anchors for leads used in the medical field.

### B. Background Art

Implantable medical devices are used for a wide variety of medical conditions. For example, a number of implantable medical devices have been commercially distributed that allow various medical agents to be controllably infused after implantation of the respective device within a patient. Such implantable medical devices may be used for the infusion of insulin, opiates, antispasmodic drugs, intrahepatic chemotherapy agents, and other therapeutic agents in a number of countries subject to the regulatory requirements of those countries. As another example, a number of implantable medical devices have been commercially distributed that allow electrical pulses or signals to be controllably delivered to a targeted tissue or nerves after implantation of the respective device within a patient. Such implantable medical devices may be used for cardiac pace making, cardiac rhythm management, treatments for congestive heart failure, implanted defibrillators, and neurostimulation. Neurostimulation encompasses a wide range of applications, such as for example, pain control, nervous tremor mitigation, incontinent treatment, epilepsy seizure reduction, and vagus nerve stimulation for clinical depression.

Implantable infusion devices typically include a central housing that includes a reservoir to hold the infusate, a septum to allow infusate to be introduced into the reservoir, an energy source to drive the infusate from the reservoir and through an outlet port, and various flow control elements. The central housing portion of the device is typically implanted in a suitable subcutaneous region with the septum positioned immediately below the skin of the patient to facilitate access to the reservoir for refilling purposes. To deliver the infusate from the reservoir, a lead in the form of a catheter is usually attached to the outlet port of the central housing to receive the infusate outflow. The distal end of the catheter is implanted within the patient adjacent to the appropriate therapy site (e.g., at a suitable intrathecal location to allow introduction of an infusate directly into the spinal fluid of the patient).

Implantable electrical stimulation devices generally include an implanted pulse generator that generates electrical pulses or signals that are transmitted to a targeted tissue or nerves through a therapy delivery element, such as a lead with electrodes. Controlled placement of the therapy delivery element is required for improved therapeutic efficacy or reduced side effects. Retaining the implanted therapy delivery element in the desired location also creates difficulties because the location may change over time as the patient moves.

Whether in a stimulation, sensing or element delivery capacity, leads are (e.g., catheter or lead having electrodes) commonly implanted along peripheral nerves, within the epidural or intrathecal space of the spinal column, and around the heart, brain, or other organs or tissue of a patient. For example, leads are often inserted such that the lead ends are adjusted precisely within the an area of placement so as to maintain an orientation, position, spacing, etc. with respect to surrounding tissue to facilitate effective treatment of one or more indications. Current lead designs, however, are often prone to movement (e.g., axially, radially and/or longitudinally) after insertion if steps are not taken to control such movement. Typically, some mechanism is employed to anchor the leads so that the therapeutic agent (e.g., infusate or electrical signal) will continue to be delivered to the appropriate site. Accordingly, various forms of anchoring structures have been utilized to discourage movement of the lead and/or the electrodes, such as to facilitate satisfactorily long functional survival time of the lead, to avoid the reprogramming or replacement of the lead to restore effective therapy, etc.

Most anchor structures for use with implantable medical device leads are either slid over the lead or clamped over the lead. In some examples, the anchor structure is secured to the lead at the same time the anchor is sutured to the fascia. For example, an anchor may be slid onto the proximal end of a lead body while maintaining the position of the lead within the body of the patient. The anchor may be secured to the lead by tying ligatures around grooves in the anchor when the anchor is sutured to the fascia or other tissue (e.g., supraspinous ligament). Existing techniques for securing anchors to the lead body can be problematic. For example, the force needed to secure the anchor to the lead is usually more than the force needed to secure the anchor to the fascia, which can lead to the lead moving axially within the anchor. Accordingly, it would be desirable to provide an improved anchor for use with medical devices.

### BRIEF SUMMARY OF THE DISCLOSURE

In one aspect, an anchor assembly for use with a medical device lead is provided. The anchor assembly comprises a holder defining a passageway therethrough, and a collapsible tube positioned partially within the passageway, the collapsible tube slidable relative to the holder to transition the anchor assembly between i) an unlocked position in which a medical device lead extending through the anchor assembly is slidable relative to the anchor assembly and ii) a locked position in which the medical device lead is fixedly coupled to the anchor assembly.

In another aspect, a system for use with a medical device lead is provided. The system comprises an anchor assembly comprising a holder defining a passageway therethrough, and a collapsible tube positioned partially within the passageway, the collapsible tube slidable relative to the holder to transition the anchor assembly between i) an unlocked position in which a medical device lead extending through the anchor assembly is slidable relative to the anchor assembly and ii) a locked position in which the medical device lead is fixedly coupled to the anchor assembly. The system further comprises a tool operable to transition the anchor assembly from the unlocked position to the locked position.

In yet another aspect, an anchor assembly for use with a medical device lead is provided. The anchor assembly comprises a body, and a door pivotably coupled to the body, the door rotatable relative to the body to transition the anchor assembly between i) an unlocked position in which a medical device lead extending through the anchor assembly is slidable relative to the anchor assembly and ii) a locked position in which the medical device lead is fixedly coupled to the anchor assembly.

In yet another aspect, an anchor assembly for use with a medical device lead is provided. The anchor assembly comprises a first component, and a second component coupled to the first component, the first component rotatable relative to the second component to transition the anchor assembly between i) an unlocked position in which a medical device lead extending through the anchor assembly is slidable relative to the anchor assembly and ii) a locked position in which the medical device lead is fixedly coupled to the anchor assembly by a shear lock formed between by the first and second components.

### BRIEF DESCRIPTION OF THE DRAWINGS

FIG. 1 is a schematic diagram of one embodiment of a stimulation system.

FIG. 2 is a perspective view of one embodiment of an anchor assembly and a lead that may be used with the system shown in FIG. 1.

FIG. 3 is a perspective view of one embodiment of a holder that may be used with the anchor assembly shown in FIG. 2.

FIG. 4 is a perspective view of one embodiment of a collapsible tube that may be used with the anchor assembly shown in FIG. 2.

FIG. 5A is a cross-sectional view of the anchor assembly shown in FIG. 2 in an unlocked position.

FIG. 5B is a cross-sectional view of the anchor assembly shown in FIG. 2 in an unlocked position.

FIGS. 6A-6G are perspective views of one embodiment of a tool that may be used to transition the anchor assembly shown in FIG. 2 from the unlocked position to the locked position.

FIGS. 7A-7C are perspective views of one embodiment of an unlocking tool that may be used to transition the anchor assembly shown in FIG. 2 from the locked position to the unlocked position.

FIGS. 8A-8D are perspective views of alternative embodiments of an anchor assembly, lead, and tool.

FIG. 9 is a perspective view of an alternative embodiment of an anchor assembly.

FIGS. 10A-10C are perspective views of alternative embodiments of an anchor assembly and lead.

FIGS. 11A and 11B are perspective views of alternative embodiments of an anchor assembly and lead.

FIGS. 12A and 12B are cross-sectional views illustrating operation of the anchor assembly shown in FIGS. 11A and 11B.

Corresponding reference characters indicate corresponding parts throughout the several views of the drawings.

### DETAILED DESCRIPTION OF THE DISCLOSURE

The present disclosure provides systems and methods for medical device anchor assemblies. An anchor assembly includes a holder defining a passageway therethrough, and a collapsible tube positioned partially within the passageway, the collapsible tube slidable relative to the holder to transition the anchor assembly between i) an unlocked position in which a medical device lead extending through the anchor assembly is slidable relative to the anchor assembly and ii) a locked position in which the medical device lead is fixedly coupled to the anchor assembly.

To aid in understanding the concepts disclosed herein, the description that follows describes examples relating to implantable medical devices of a spinal cord stimulation (SCS) system. However, it is to be understood that, while embodiments of the anchor assembly disclosed herein are well suited for applications in SCS, the disclosure in its broadest aspects may not be so limited. Rather, the disclosure may be used with any type of implantable therapy delivery system with one or more therapy delivery elements. For example, the present disclosure may be used as part of a pacemaker, a defibrillator, a cochlear stimulator, a retinal stimulator, a stimulator configured to produce coordinated limb movement, a cortical stimulator, a deep brain stimulator, peripheral nerve stimulator, microstimulator, or in any other neural stimulator configured to treat urinary incontinence, sleep apnea, shoulder subluxation, headache, etc.

Accordingly, the anchor assembly described herein may be used with one or more therapy delivery elements including an electrical lead having one or more electrodes to deliver pulses or signals to a respective target tissue site in a patient. Additionally or alternatively, the anchor assembly described herein may be used with one or more therapy delivery elements including an electrical lead including sensing electrodes to sense physiological parameters (e.g., blood pressure, temperature, cardiac activity, etc.) at a target tissue site within a patient.

In accordance with some embodiments, the anchor assembly described herein may be used with one or more therapy delivery elements comprising a fluid delivery conduit, such as a catheter, including an inner lumen that is placed to deliver a fluid, such as pharmaceutical agents, insulin, pain relieving agents, gene therapy agents, or the like from a fluid delivery device (e.g., a fluid reservoir and/or pump) to a respective target tissue site in a patient.

In the various embodiments contemplated by this disclosure, therapy may include stimulation therapy, sensing or monitoring of one or more physiological parameters, fluid delivery, and the like. A therapy delivery element (also referred to as a lead) may include pacing or defibrillation leads, stimulation leads, sensing leads, fluid delivery conduit, extensions for any of the above, or combinations thereof. A target tissue site may refer generally to the target site for implantation of a therapy delivery element, regardless of the type of therapy.

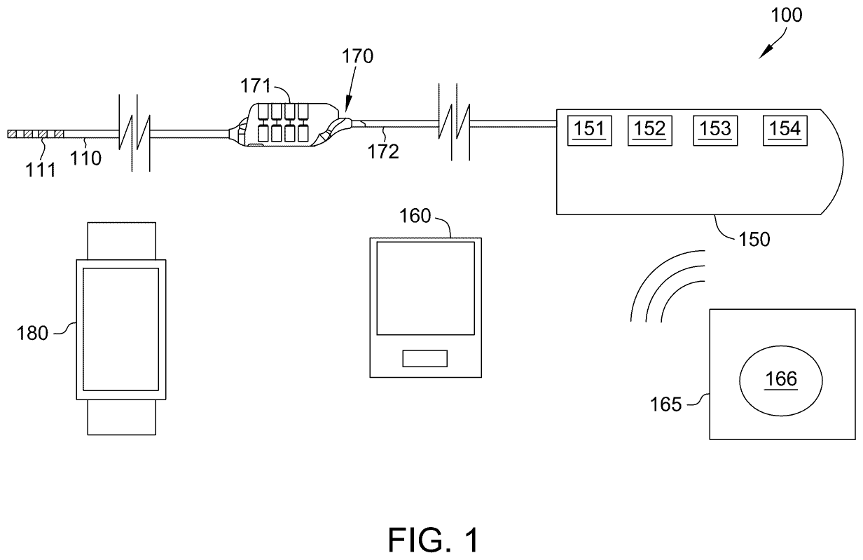

FIG. 1 is a schematic diagram of one embodiment of a stimulation system 100. Stimulation system 100 may be, for example, a spinal cord stimulation (SCS) system. Stimulation system 100 generates electrical pulses for application to tissue of a patient to treat one or more disorders of the patient. System 100 includes an implantable pulse generator (IPG) 150 that is adapted to generate electrical pulses for application to tissue of a patient. Examples of commercially available implantable pulse generators include the PROCLAIM XR™ and INFINITY™ implantable pulse generators (available from ABBOTT, PLANO TX). Commercially available IPGs may be adapted (using suitable software instructions, programmable parameters, logic circuits, other circuits, and/or the like) according to the disclosures in this application. Alternatively, system 100 may include an external pulse generator (EPG) positioned outside the patient's body. IPG 150 typically includes a metallic housing (or can) that encloses a controller 151, pulse generating circuitry 152, a battery 153, far-field and/or near field communication circuitry 154 (e.g., BLUETOOTH communication circuitry), sensing circuitry 155, and other appropriate circuitry and components of the device. Controller 151 typically includes a microcontroller or other suitable processor for controlling the various other components of the device. Software code is typically stored in memory of IPG 150 for execution by the microcontroller or processor to control the various components of the device.

IPG 150 may include one or more attached extension components 170 or be connected to one or more separate extension components 170. Alternatively, one or more stimulation leads 110 may be connected directly to IPG 150. Within IPG 150, electrical pulses are generated by pulse generating circuitry 152 and are provided to switching circuitry. The switching circuit connects to output wires, metal ribbons, traces, lines, or the like (not shown) from the internal circuitry of pulse generator 150 to output connectors (not shown) of pulse generator 150 which are typically contained in the “header” structure of pulse generator 150. Commercially available ring/spring electrical connectors are frequently employed for output connectors of pulse generators (e.g., “Bal-Seal” connectors). The terminals of one or more stimulation leads 110 are inserted within connector portion 171 for electrical connection with respective connectors or directly within the header structure of pulse generator 150. Thereby, the pulses originating from IPG 150 are conducted to electrodes 111 through wires contained within the lead body of lead 110. The electrical pulses are applied to tissue of a patient via electrodes 111.

For implementation of the components within IPG 150, a processor and associated charge control circuitry for an implantable pulse generator is described in U.S. Pat. No. 7,571,007, entitled “SYSTEMS AND METHODS FOR USE IN PULSE GENERATION,” which is incorporated herein by reference. Circuitry for recharging a rechargeable battery of an implantable pulse generator using inductive coupling and external charging circuits are described in U.S. Pat. No. 7,212,110, entitled “IMPLANTABLE DEVICE AND SYSTEM FOR WIRELESS COMMUNICATION,” which is incorporated herein by reference.

An example and discussion of “constant current” pulse generating circuitry is provided in U.S. Patent Publication No. 2006/0170486 entitled “PULSE GENERATOR HAVING AN EFFICIENT FRACTIONAL VOLTAGE CONVERTER AND METHOD OF USE,” which is incorporated herein by reference. One or multiple sets of such circuitry may be provided within IPG 150. Different pulses on different electrodes may be generated using a single set of pulse generating circuitry using consecutively generated pulses according to a “multi-stimset program” as is known in the art. Alternatively, multiple sets of such circuitry may be employed to provide pulse patterns that include simultaneously generated and delivered stimulation pulses through various electrodes of one or more stimulation leads as is also known in the art. Various sets of parameters may define the pulse characteristics and pulse timing for the pulses applied to various electrodes as is known in the art. Although constant current pulse generating circuitry is contemplated for some embodiments, any other suitable type of pulse generating circuitry may be employed such as constant voltage pulse generating circuitry.

Stimulation lead(s) 110 may include a lead body of insulative material about a plurality of conductors within the material that extend from a proximal end of lead 110 to its distal end. The conductors electrically couple a plurality of electrodes 111 to a plurality of terminals (not shown) of lead 110. The terminals are adapted to receive electrical pulses and the electrodes 111 are adapted to apply stimulation pulses to tissue of the patient. Also, sensing of physiological signals may occur through electrodes 111, the conductors, and the terminals. Additionally or alternatively, various sensors (not shown) may be located near the distal end of stimulation lead 110 and electrically coupled to terminals through conductors within the lead body 172. Stimulation lead 110 may include any suitable number and type of electrodes 111, terminals, and internal conductors.

External controller device 160 is a device that permits the operations of IPG 150 to be controlled by a user after IPG 150 is implanted within a patient. Also, multiple controller devices may be provided for different types of users (e.g., the patient or a clinician). Controller device 160 can be implemented by utilizing a suitable handheld processor-based system that possesses wireless communication capabilities. Software is typically stored in memory of controller device 160 to control the various operations of controller device 160. The interface functionality of controller device 160 is implemented using suitable software code for interacting with the user and using the wireless communication capabilities to conduct communications with IPG 150. One or more user interface screens may be provided in software to allow the patient and/or the patient's clinician to control operations of IPG 150 using controller device 160. In some embodiments, commercially available devices such as APPLE IOS devices are adapted for use as controller device 160 by include one or more “apps” that communicate with IPG 150 using, for example, BLUETOOTH communication.

Controller device 160 may provide one or more user interfaces to allow the user to operate IPG 150 according to one or more stimulation programs to treat the patient's disorder(s). Each stimulation program may include one or more sets of stimulation parameters including pulse amplitude, pulse width, pulse frequency or inter-pulse period, pulse repetition parameter (e.g., number of times for a given pulse to be repeated for respective stimset during execution of program), etc.

Controller device 160 may permit programming of IPG 150 to provide a number of different stimulation patterns or therapies to the patient as appropriate for a given patient and/or disorder. Examples of different stimulation therapies include conventional tonic stimulation (continuous train of stimulation pulses at a fixed rate), BurstDR stimulation (burst of pulses repeated at a high rate interspersed with quiescent periods with or without duty cycling), “high frequency” stimulation (e.g., a continuous train of stimulation pulses at 10,000 Hz), noise stimulation (series of stimulation pulses with randomized pulse characteristics such as pulse amplitude to achieve a desired frequency domain profile). Any suitable stimulation pattern or combination thereof can be provided by IPG 150 according to some embodiments. Controller device 160 communicates the stimulation parameters and/or a series of pulse characteristics defining the pulse series to be applied to the patient to IPG 150 to generate the desired stimulation therapy.

Examples of suitable therapies include tonic stimulation (in which a fixed frequency pulse train) is generated, burst stimulation (in which bursts of multiple high frequency pulses) are generated which in turn are separated by quiescent periods, “high frequency” stimulation, multi-frequency stimulation, noise stimulation. Examples of suitable therapies include tonic stimulation (in which a fixed frequency pulse train) is generated, burst stimulation (in which bursts of multiple high frequency pulses) are generated which in turn are separated by quiescent periods, “high frequency” stimulation, multi-frequency stimulation, and noise stimulation. Descriptions of respective neurostimulation therapies are provided in the following publications: (1) Schu S., Slotty P. J., Bara G., von Knop M., Edgar D., Vesper J. A Prospective, Randomised, Double-blind, Placebo-controlled Study to Examine the Effectiveness of Burst Spinal Cord Stimulation Patterns for the Treatment of Failed Back Surgery Syndrome. Neuromodulation 2014; 17:443-450; (2) Al-Kaisy A1, Van Buyten J P, Smet I, Palmisani S, Pang D, Smith T. 2014. Sustained effectiveness of 10 kHz high-frequency spinal cord stimulation for patients with chronic, low back pain: 24-month results of a prospective multicenter study. Pain Med. 2014 March; 15(3):347-54; and (3) Sweet, Badjatiya, Tan D1, Miller. Paresthesia-Free High-Density Spinal Cord Stimulation for Postlaminectomy Syndrome in a Prescreened Population: A Prospective Case Series. Neuromodulation. 2016 April; 19(3):260-7. Noise stimulation is described in U.S. Pat. No. 8,682,441B2. Burst stimulation is described in U.S. Pat. No. 8,224,453 and U.S. Published Application No. 20060095088. A “coordinated reset” pulse pattern is applied to neuronal subpopulation/target sites to desynchronize neural activity in the subpopulations. Coordinated reset stimulation is described, for example, by Peter A. Tass et al in COORDINATED RESET HAS SUSTAINED AFTER EFFECTS IN PARKINSONIAN MONKEYS, Annals of Neurology, Volume 72, Issue 5, pages 816-820, November 2012, which is incorporated herein by reference. The electrical pulses in a coordinated reset pattern are generated in bursts of pulses with respective bursts being applied to tissue of the patient using different electrodes in a time-offset manner. The time-offset is selected such that the phase of the neural-subpopulations are reset in a substantially equidistant phase-offset manner. By resetting neuronal subpopulations in this manner, the population will transition to a desynchronized state by the interconnectivity between the neurons in the overall neuronal population. All of these references are incorporated herein by reference.

For implementation of the components within IPG 150, a processor and associated charge control circuitry for an implantable pulse generator is described in U.S. Pat. No. 7,571,007, entitled “SYSTEMS AND METHODS FOR USE IN PULSE GENERATION,” which is incorporated herein by reference. Circuitry for recharging a rechargeable battery of an implantable pulse generator using inductive coupling and external charging circuits are described in U.S. Pat. No. 7,212,110, entitled “IMPLANTABLE DEVICE AND SYSTEM FOR WIRELESS COMMUNICATION” which is incorporated herein by reference.

IPG 150 modifies its internal parameters in response to the control signals from controller device 160 to vary the stimulation characteristics of stimulation pulses transmitted through stimulation lead 110 to the tissue of the patient. Neurostimulation systems, stimsets, and multi-stimset programs are discussed in PCT Publication No. WO 2001/093953, entitled “NEUROMODULATION THERAPY SYSTEM,” and U.S. Pat. No. 7,228,179, entitled “METHOD AND APPARATUS FOR PROVIDING COMPLEX TISSUE STIMULATION PATTERNS,” which are incorporated herein by reference.

External charger device 165 may be provided to recharge battery 153 of IPG 150 according to some embodiments when IPG 150 includes a rechargeable battery. External charger device 165 includes a power source and electrical circuitry (not shown) to drive current through coil 166. The patient places primary coil 166 against the patient's body immediately above the secondary coil (not shown), i.e., the coil of the implantable medical device. Primary coil 166 and the secondary coil may be aligned in a coaxial manner by the patient for efficiency of the coupling between the primary and secondary coils. In operation during a charging session, external charger device 165 generates an AC-signal to drive current through coil 166 at a suitable frequency. Assuming that primary coil 166 and secondary coil are suitably positioned relative to each other, the secondary coil is disposed within the magnetic field generated by the current driven through primary coil 166. Current is then induced by a magnetic field in the secondary coil. The current induced in the coil of the implantable pulse generator is rectified and regulated to recharge the battery of IPG 150. IPG 150 may also communicate status messages to external charging device 165 during charging operations to control charging operations. For example, IPG 150 may communicate the coupling status, charging status, charge completion status, etc.

System 100 may include external wearable device 180 such as a smartwatch or health monitor device. Wearable device may be implemented using commercially available devices such as FITBIT VERSA SMARTWATCH™, SAMSUNG GALAXY SMARTWATCH™, and APPLE WATCH™ devices with one or more apps or appropriate software to interact with IPG 150 and/or controller device 160. In some embodiments, wearable device 180, controller device 160, and IPG 150 conduct communications using BLUETOOTH communications.

Wearable device 180 monitors activities of the patient and/or senses physiological signals. Wearable device 180 may track physical activity and/or patient movement through accelerometers. Wearable device 180 may monitory body temperature, heart rate, electrocardiogram activity, blood oxygen saturation, and/or the like. Wearable device 180 may monitor sleep quality or any other relevant health related activity.

Wearable device 180 may provide one or more user interface screens to permit the patient to control or otherwise interact with IPG 150. For example, the patient may increase or decrease stimulation amplitude, change stimulation programs, turn stimulation on or off, and/or the like using wearable device 180. Also, the patient may check the battery status of other implant status information using wearable device 180.

Wearable device 180 may include one or more interface screens to receive patient input. In some embodiments, wearable device 180 and/or controller device 160 are implemented (individually or in combination) to provide an electronic patient diary function. The patient diary function permits the patient to record on an ongoing basis the health status of the patient and the effectiveness of the therapy for the patient. In some embodiments as discussed herein, wearable device 180 and/or controller device 160 enable the user to indicate the current activity of the patient, the beginning of an activity, the completion of an activity, the ease or quality of patient's experience with a specific activity, the patient's experience of pain, the patient's experience of relief from pain by the stimulation, or any other relevant indication of patient health by the patient.

Since IPG 150 is typically located remotely from a target location for therapy, one or more stimulation leads 110 and/or lead body 172 are typically routed through a pathways subcutaneously formed along the torso of the patient to a subcutaneous pocket where IPG 150 is located.

Stimulation leads 110 and/or lead body 172 are typically fixed in place near a location selected by a clinician using one or more anchors (not shown in FIG. 1), such as in an epidural space. The anchor may be positioned on stimulation leads 110 and/or lead body 172 in a wide variety of locations and orientations to accommodate individual anatomical differences and the preferences of the clinician. The anchor may then be affixed to tissue using fasteners, such as for example, one or more sutures, staples, screws, or other fixation devices. The tissue to which the anchor is affixed may include subcutaneous fascia layer, bone, or some other type of tissue. Securing the anchor to tissue in this manner prevents or reduces the chance that stimulation leads 110 and/or lead body 172 will become dislodged or will migrate in an undesired manner.

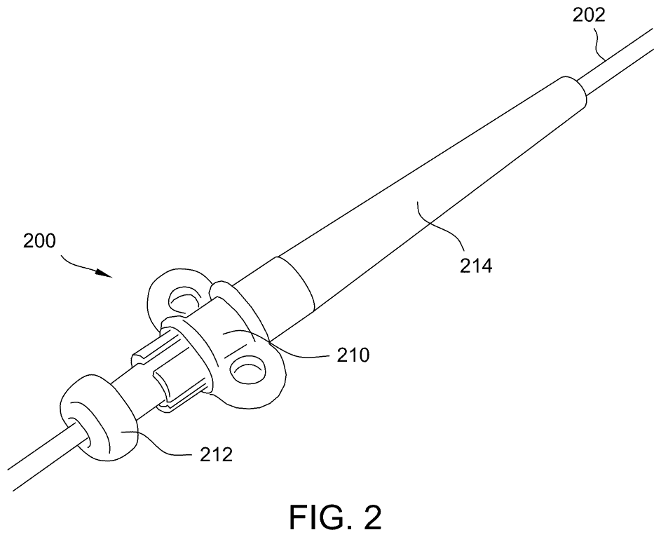

FIG. 2 is a perspective view of one embodiment of an anchor assembly 200 and a lead 202. Anchor assembly 200 may be used with, for example, system 100 (shown in FIG. 1). In this embodiment, anchor assembly 200 includes a holder 210, a collapsible tube 212, and a flexible member 214. Holder 210 is coupled between collapsible tube 212 and flexible member 214. In some embodiments, flexible member 214 is omitted.

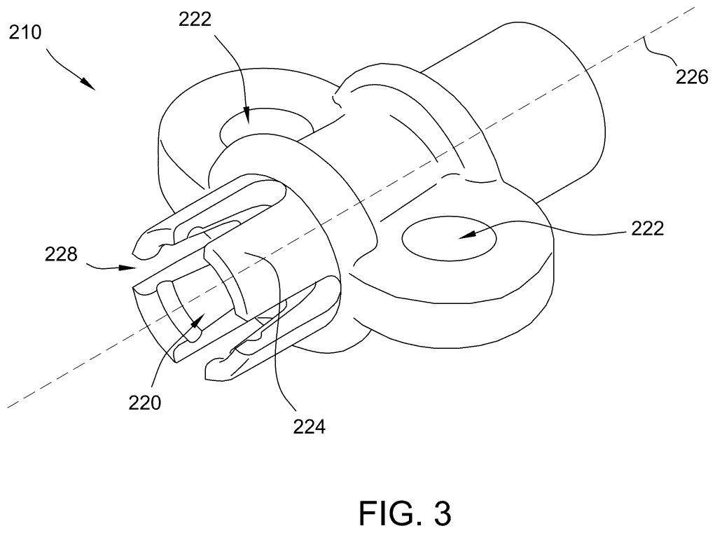

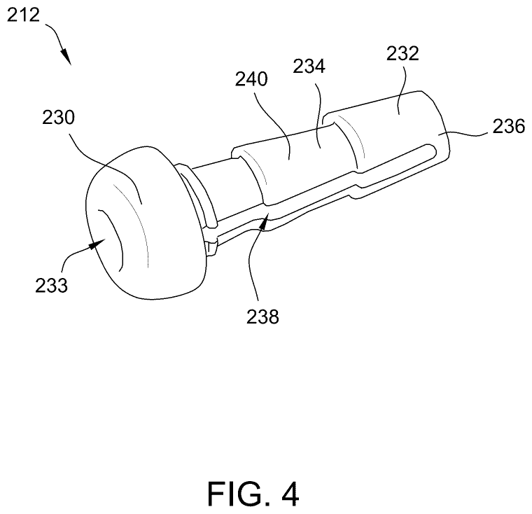

FIG. 3 is a perspective view of holder 210, and FIG. 4 is a perspective view of collapsible tube 212. As shown, holder 210 has a generally cylindrical shape that defines a passageway 220 therethrough. Further, holder 210 defines two fixture holes 222 that may be used when attaching anchor assembly 200 to tissue. For example, sutures may be threaded through fixture holes 222.

Holder 210 further includes a plurality of prongs 224 that extend axially along a longitudinal axis 226 of holder 210 and that are arranged circumferentially about longitudinal axis 226. Prongs 224 are separated from one another by gaps 228. This enables prongs 224 to flex radially inwardly and outwardly with respect to longitudinal axis 226. In this embodiment, holder 210 includes four prongs 224. Alternatively, holder 210 may include any suitable number of prongs 224.

Referring to FIG. 4, collapsible tube 212 includes an annular head 230 and a body 232 that define a tube passageway 233 therethrough. Body 232 includes a plurality of struts 234 that extend axially between annular head 230 and an annular base 236. Struts 234 are separated from one another by gaps 238. This enables struts 234 to flex radially inwardly and outwardly. In this embodiment, collapsible tube 212 includes three struts 234. Alternatively, collapsible tube 212 may include any suitable number of struts 234. Further, struts 234 each include one or more ramped surfaces 240. As described below, ramped surfaces 240 facilitate coupling anchor assembly 200 to lead 202.

Referring back to FIG. 2, in anchor assembly 200, collapsible tube 212 is coupled to holder 210 such that a portion of collapsible tube 212 extends through passageway 220. Further, as described in detail herein, collapsible tube 212 is slidable relative to holder 210 between a locked position and an unlocked position of anchor assembly 200. In general, while lead 202 extends through anchor assembly 200 (i.e., through tube passageway 233), anchor assembly 200 is slidable along lead 202 when anchor assembly 200 is in the unlocked position. In contrast, when anchor assembly 200 is in the locked position, anchor assembly 200 is fixedly coupled to lead 202 (i.e., lead 202 cannot slide relative to anchor assembly 200). In FIG. 2, collapsible tube 212 is shown in the unlocked position of anchor assembly 200.

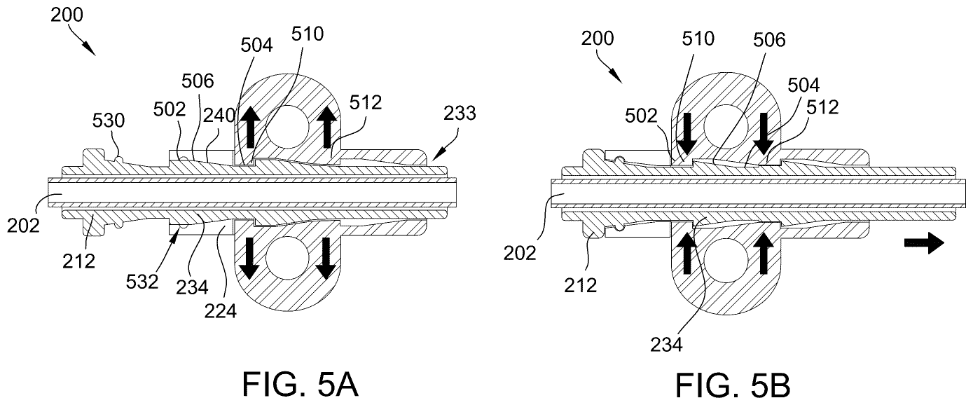

FIG. 5A is a cross-sectional view of anchor assembly 200 with collapsible tube 212 in the unlocked position of anchor assembly 200, and FIG. 5B is a cross-sectional view of anchor assembly 200 with collapsible tube 212 in the locked position anchor assembly 200.

As shown in FIG. 5A, ramped surfaces 240 include a first portion 502, a second portion 504, and a tapered portion 506 extending between first and second portions 502 and 504. First portion 502 is radially outward from second portion 504. Further, as shown in FIG. 5A, in this embodiment, holder 210 includes a first annular shoulder 510 and a second annular shoulder 512.

In the unlocked position (shown in FIG. 5A), second portions 504 of ramped surfaces 240 contact first and second annular shoulders 510 and 512. Ramped surfaces 240 and first and second annular shoulders 510 and 512 are dimensioned such that first and second annular shoulders 510 and 512 do not compress ramped surfaces 240 radially inward. Accordingly, lead 202 is capable of sliding within tube passageway 233.

To transition from the unlocked position to the locked position, collapsible tube 212 is pushed or slid further into holder 210 (i.e., collapsible tube 212 is pushed to the right relative to holder 210 in the views of FIGS. 5A and 5B).

As anchor assembly 200 transitions from the unlocked position to the locked position, first and second annular shoulders 510 and 512 slide along tapered portions 506 until first and second annular shoulders 510 contact first portions 502. As shown in FIGS. 5A and 5B, in the unlocked position, first portions 502 are radially outward of first and second annular shoulders 510. Accordingly, when transitioning from the unlocked position to the locked position, first portions 502 are urged radially inward, urging struts 234 radially inward. This results in struts 234 compressing on lead 202, which fixes the position of lead 202 relative to anchor assembly 200.

As shown in FIGS. 5A and 5B, in some embodiments, collapsible tube 212 includes one or more protrusions 530. Protrusions 530 are sized to engage corresponding grooves 532 defined in prongs 224 when anchor assembly 200 is in the locked position. This further facilitates securing collapsible tube 212 in the locked position.

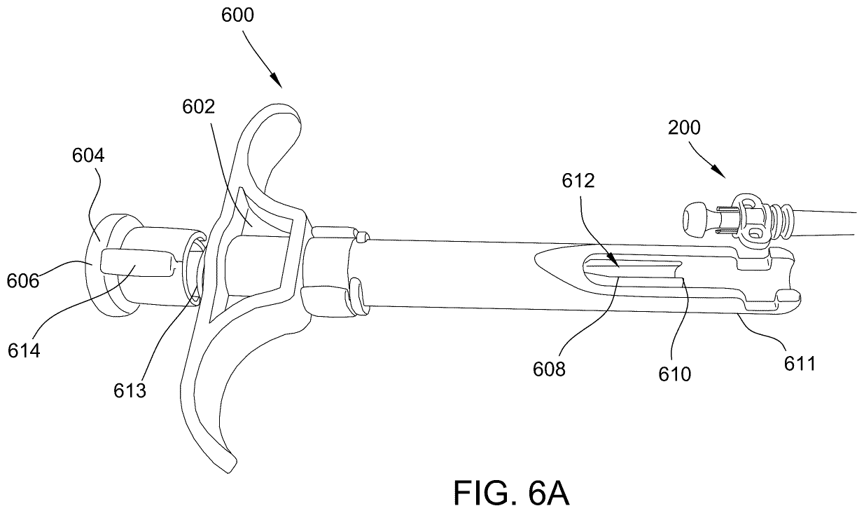

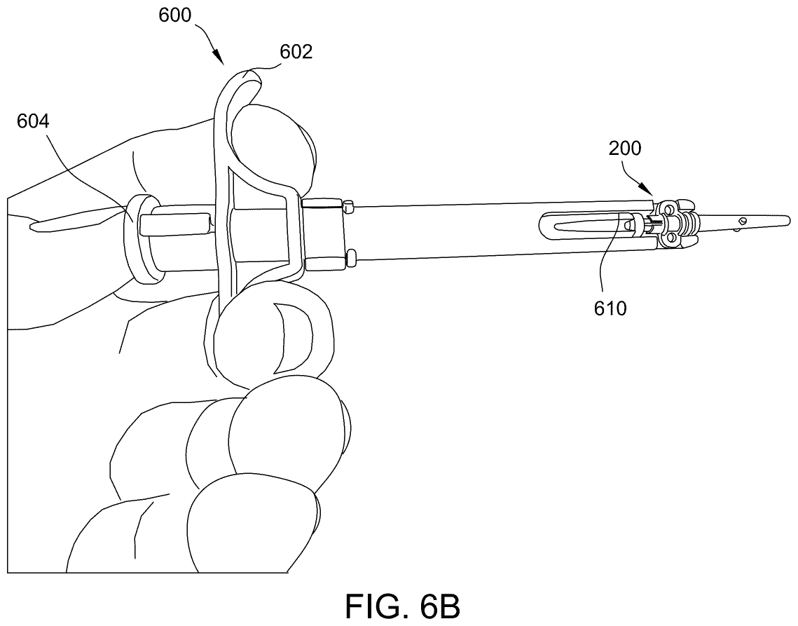

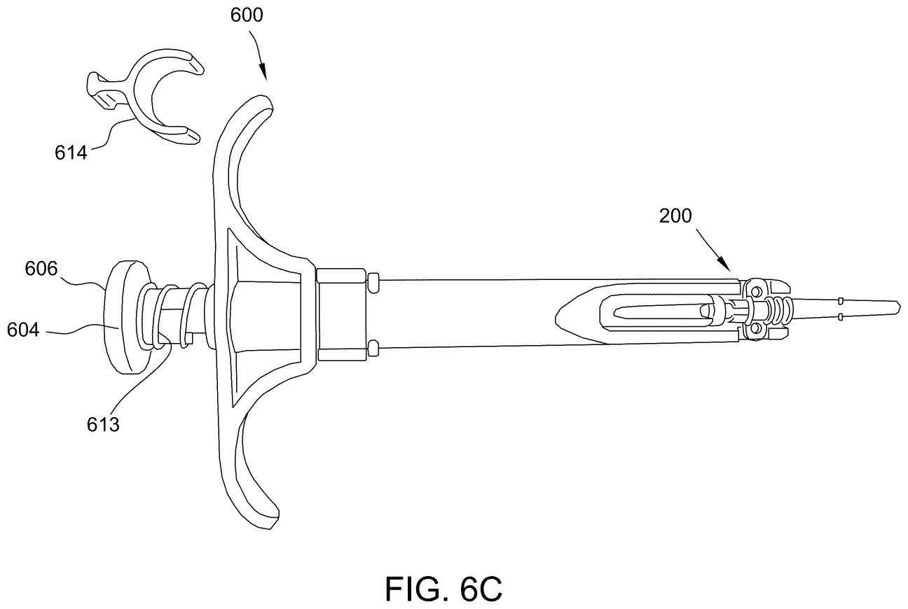

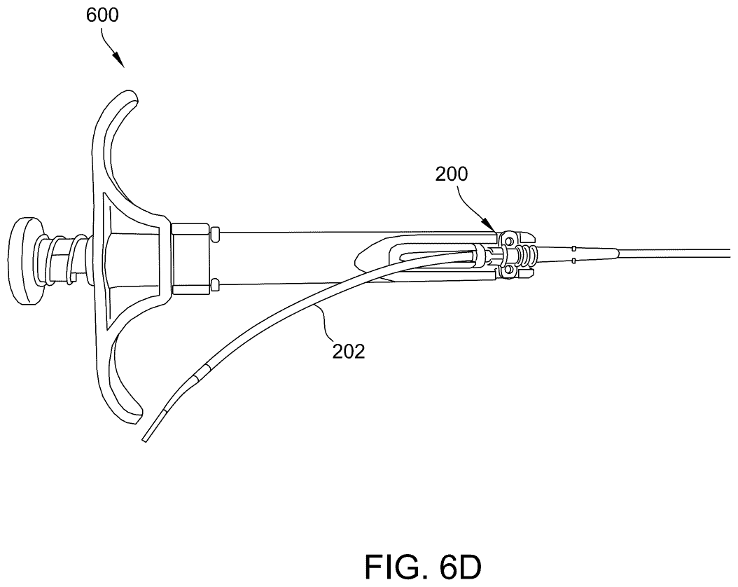

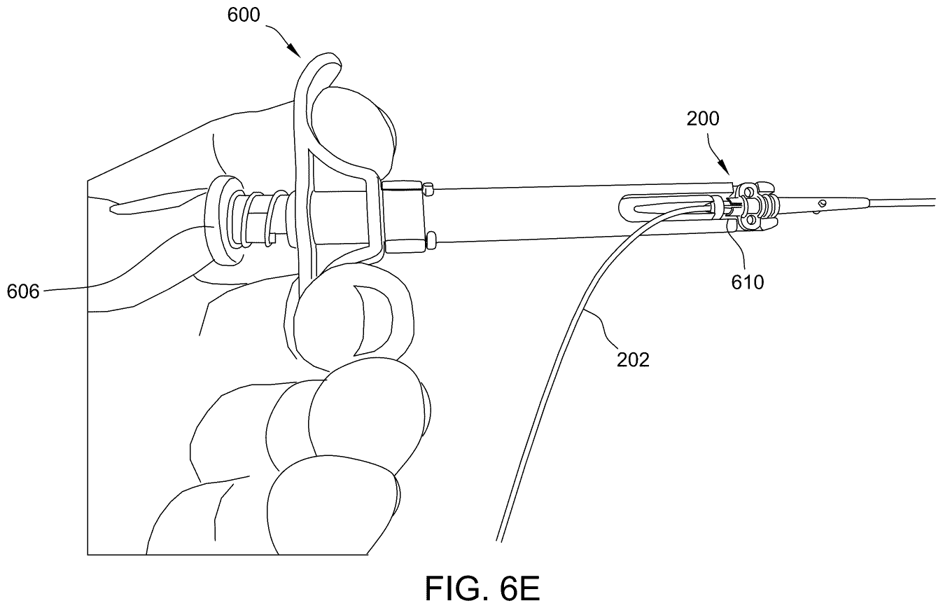

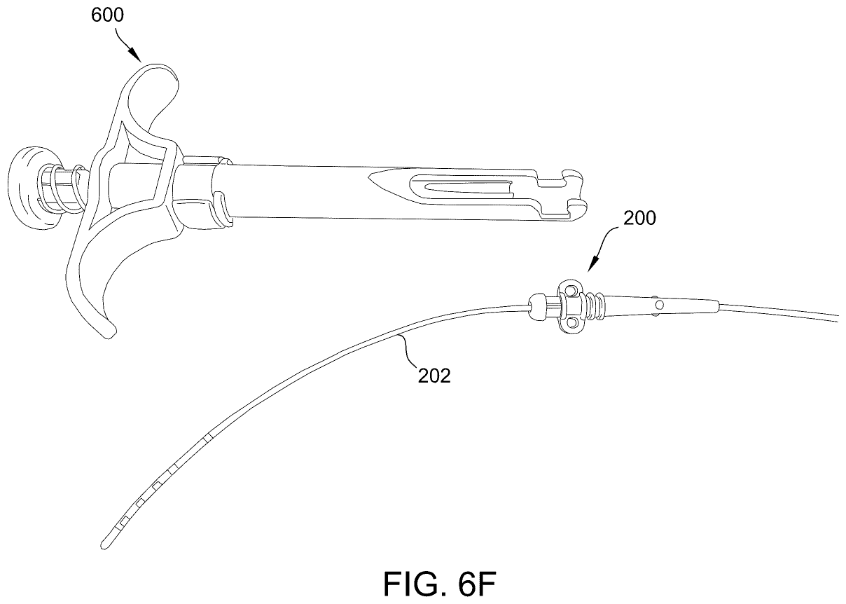

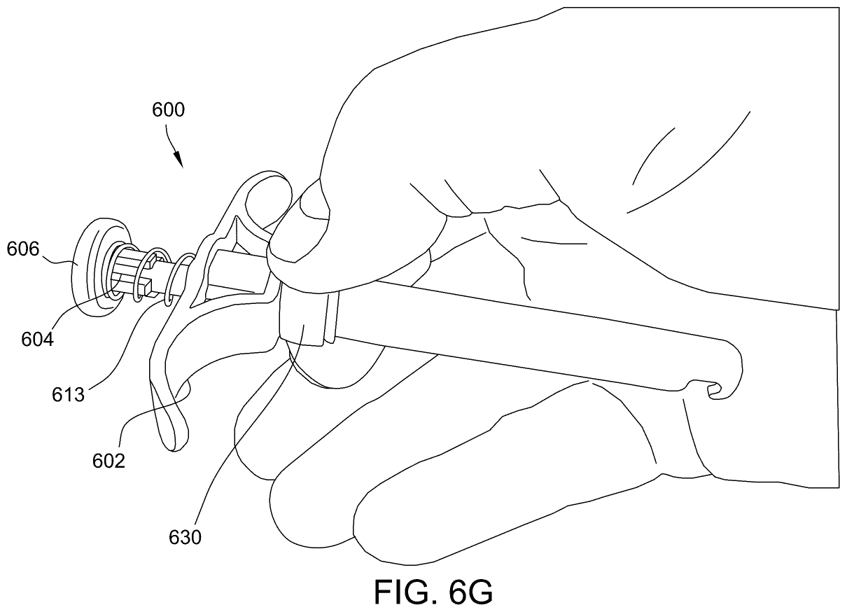

FIGS. 6A-6G are perspective views of one embodiment of a tool 600 that may be used to transition anchor assembly 200 from the unlocked position to the locked position. Tool 600 includes a handle 602 and a plunger 604 slidable within handle 602. Plunger 604 extends from a knob 606 to an anchor interface 608. Anchor interface 608 includes an engagement end 610 and defines a groove 612. Further, handle 602 defines a slot 611 such that anchor interface 608 is visible/exposed. A biasing member 613 (e.g., a spring) biases the knob 606 in a direction away from handle 602 (i.e., to the left in FIGS. 6A-6G). Further, a locking clip 614 is coupled to plunger 604 proximate knob 606. Locking clip 614 may be, for example, a c-shaped clip.

An example operation of tool 600 is described herein. Those of skill in the art will appreciate that tool 600 may be used in alternative manners. In FIG. 6A, tool 600 is shown in an initial state. To use tool 600, as shown in FIG. 6A, anchor assembly 200 (in the unlocked position) is inserted into slot 611 by a user while tool 600 is in the initial state.

Subsequently, as shown in FIG. 6B, plunger 604 is depressed by the user until engagement end 610 contacts anchor assembly 200. This is referred to herein as a primed state. In this embodiment, once tool 600 reaches the primed state, plunger 604 engages handle 602 to secure tool 600 in the primed state. For example, plunger 604 may include a protrusion that engages a corresponding groove defined within handle 602. Notably, in the primed state, anchor assembly 200 is still in the unlocked position.

As shown in FIG. 6C, with tool 600 in the primed state, locking clip 614 is removed from plunger 604 by the user. This enables knob 606 to be further advanced towards handle 602. Further, as shown in FIG. 6D, lead 202 is advanced through anchor assembly 200 by the user while anchor assembly 200 is in the unlocked position and tool 600 is in the primed state.

Once lead 202 is advanced to a desired position (relative to anchor assembly 200), knob 606 is further advanced by the user towards handle 602. This causes engagement end 610 (which is in contact with anchor assembly 200) to push collapsible tube 212 further into holder 210, transitioning anchor assembly 200 from the unlocked position to the locked position, and fixedly coupling anchor assembly 200 to lead 202. This is shown in FIG. 6E.

When the user releases knob 606, plunger 604 returns to the primed state, while anchor assembly 200 remains in the locked position. At this point, anchor assembly 200 may be removed from tool 600 by the user, as shown in FIG. 6F.

As shown in FIG. 6G, the user may press an actuator 630 on handle 602 to return tool 600 to the initial state. For example, pressing actuator 630 causes the protrusion on plunger 604 to disengage from the corresponding groove in handle 604, and biasing member 613 urges knob 606 away from handle 602, returning tool 600 to the initial state. At this point, locking clip 614 may be again coupled to plunger 604. Thus, tool 600 is effectively reset for use with additional anchor assemblies.

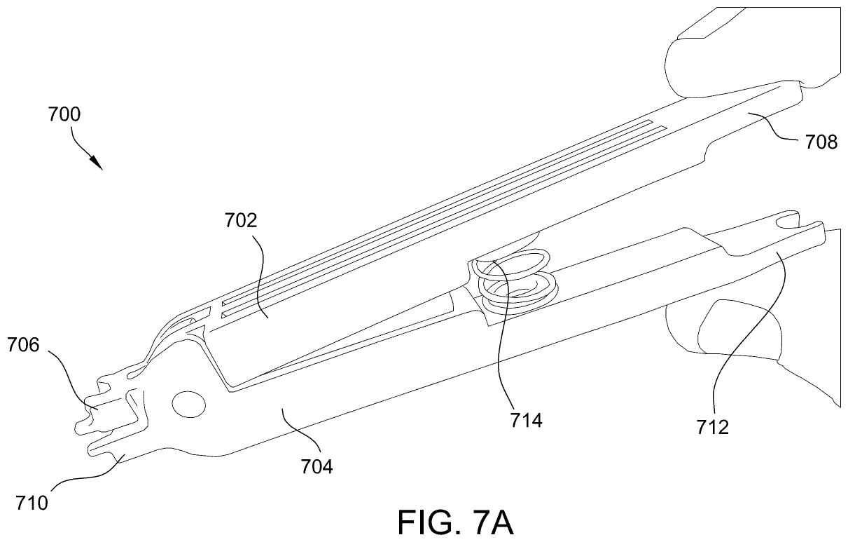

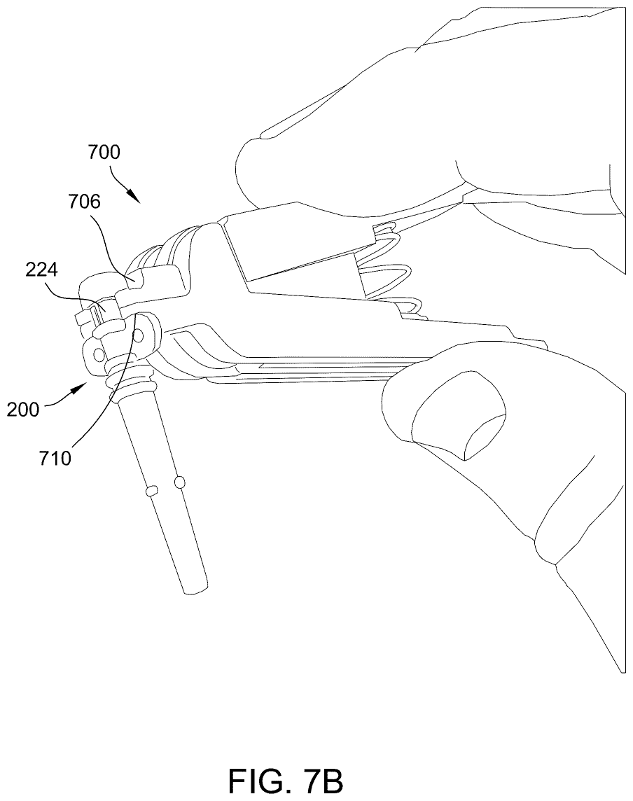

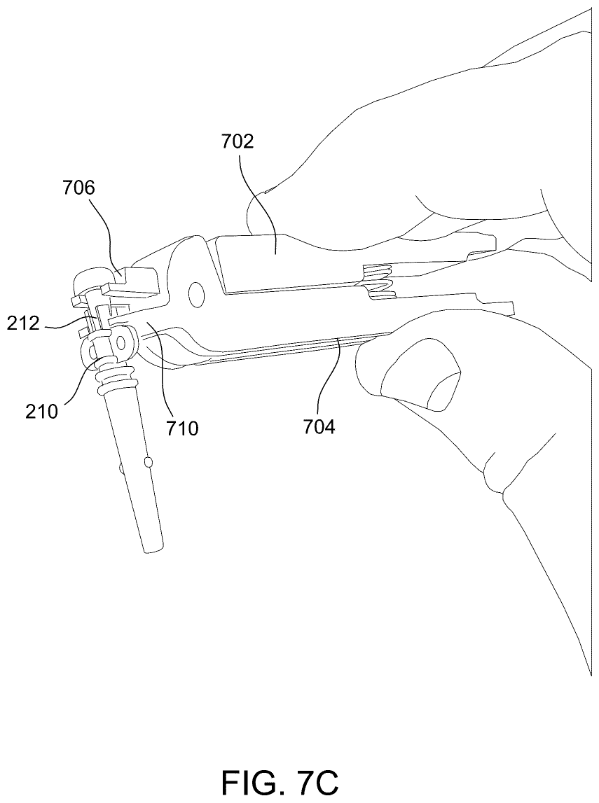

FIGS. 7A-7C are perspective views of one embodiment of an unlocking tool 700 that may be used to transition anchor assembly 200 from the locked position to the unlocked position. That is, unlocking tool 700 may be used to decouple anchor assembly 200 from lead 202.

In this embodiment, as shown in FIG. 7A, unlocking tool 700 includes a first lever arm 702 rotatably coupled to a second lever arm 704. First lever arm 702 extends between a first engagement end 706 and a first handle end 708. Similarly, second lever arm 704 extends between a second engagement end 710 and a second handle end 712.

A spring member 714 extending between first and second lever arms 702 and 704 biases first and second handle ends 708 and 712 away from one another and biases first and second engagement ends 706 and 710 toward one another.

As shown in FIG. 7B, to transition anchor assembly 200 to the unlocked position, the user positions first and second engagement ends 706 and 710 around prongs 224. Then, as shown in FIG. 7C, the user squeezes together first and second lever arms 702 and 704. This causes first engagement end 706 and second engagement end 710 to push collapsible tube 212 and holder 210 away from one another, which causes anchor assembly to transition from the locked position to the unlocked position. At that point, lead 202 may be removed from anchor assembly 200.

Those of skill in the art will appreciate that the embodiments shown in FIGS. 2-7C are merely examples, and that other anchor assemblies and tools fall within the spirit and scope of the present disclosure.

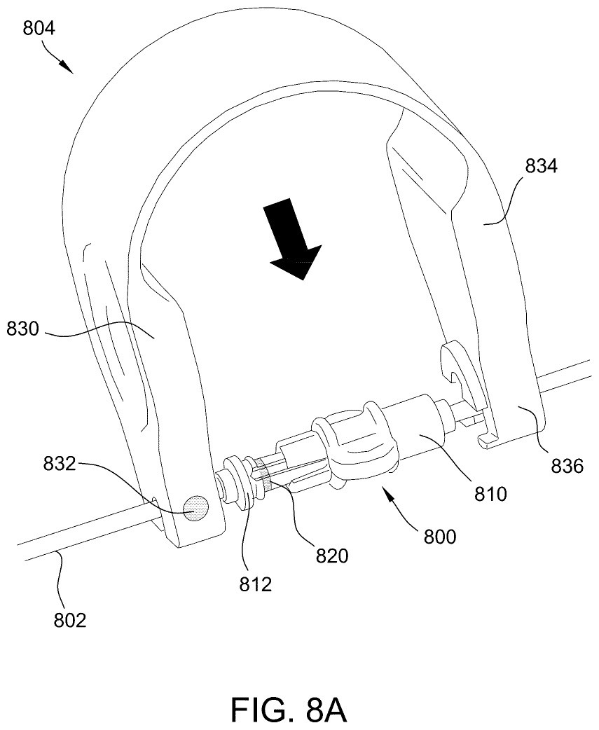

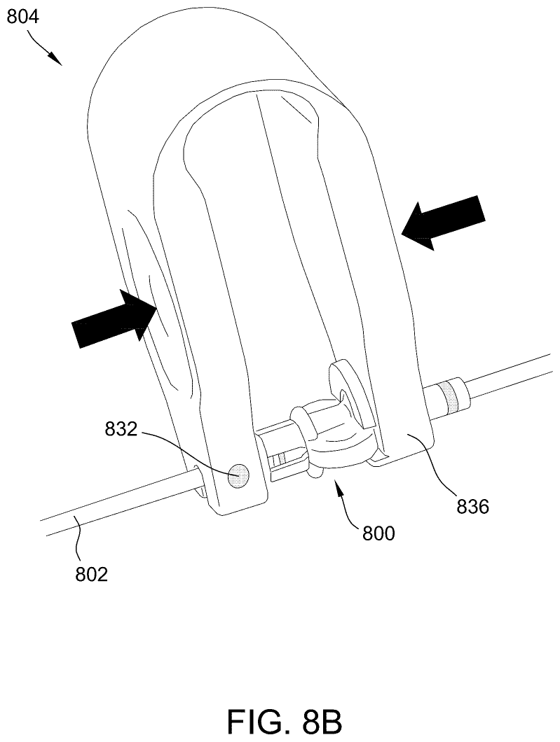

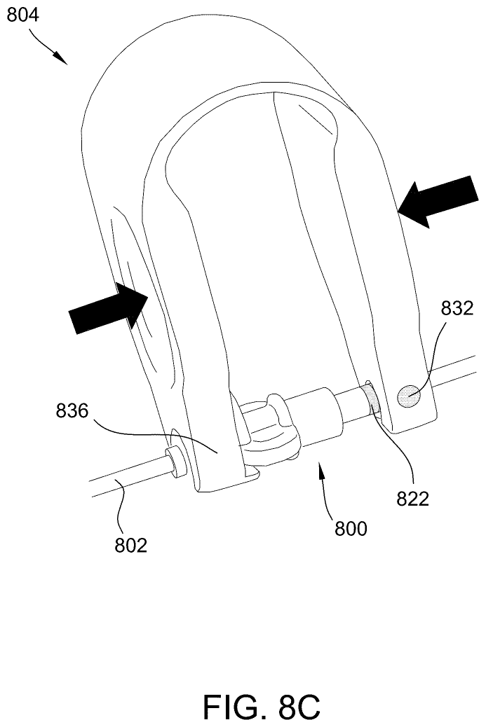

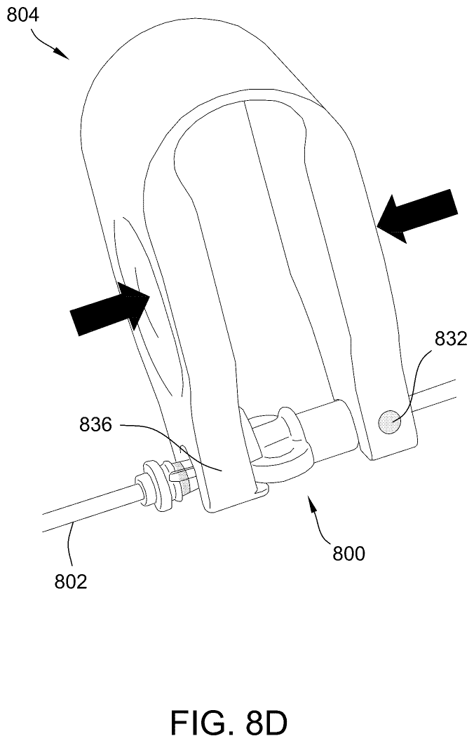

For example, FIGS. 8A-8D are perspective views of another embodiment of an anchor assembly 800, lead 802, and tool 804. Similar to anchor assembly 200, anchor assembly 800 includes a holder 810 and a collapsible tube 812. Unless otherwise indicated, holder 810 and collapsible tube 812 function similarly to holder 210 and collapsible tube 212.

In this embodiment, collapsible tube 812 includes a unlock marker band 820 that is visible when the anchor assembly 800 is in the unlocked position (shown in FIG. 8A) and at least one lock marker band 822 that is visible when the anchor assembly 800 is in the unlocked position (shown in FIG. 8C).

Tool 804 is a generally c-shaped component that can be used to selectively lock and unlock anchor assembly 800. Tool 804 includes a first arm 830 with a first engagement end 832 and a second arm 834 with a second engagement end 836.

As shown in FIGS. 8A and 8B, to lock anchor assembly 800, first and second engagement ends 832 and 836 are positioned proximate collapsible tube 812 and holder 810 by the user. Then, the user squeezes first and second arms 830 and 834 together, pushing collapsible tube 812 into holder 810, and putting anchor assembly 800 in the locked position.

As shown in FIGS. 8C and 8D, to unlock anchor assembly 800, first and second engagement ends 832 and 836 are positioned proximate collapsible tube 812 and holder 810 by the user. Notably, tool 600 is placed in a reverse orientation to unlock anchor assembly 800 (i.e., first and second engagement ends 832 and 836 have switched places). Then, the user squeezes first and second arms 830 and 834 together, pushing collapsible tube 812 out of holder 810, and putting anchor assembly 800 in the unlocked position.

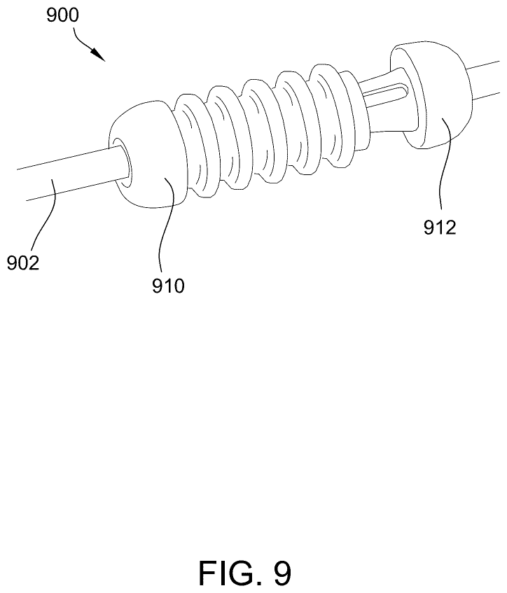

FIG. 9 is a perspective view of another embodiment of an anchor assembly 900 and lead 902. Similar to anchor assembly 200, anchor assembly 900 includes a holder 910 and a collapsible tube 912. Unless otherwise indicated, holder 910 and collapsible tube 912 function similarly to holder 910 and collapsible tube 912. As shown in FIG. 9, holder 910 has a ribbed shaped in this embodiment.

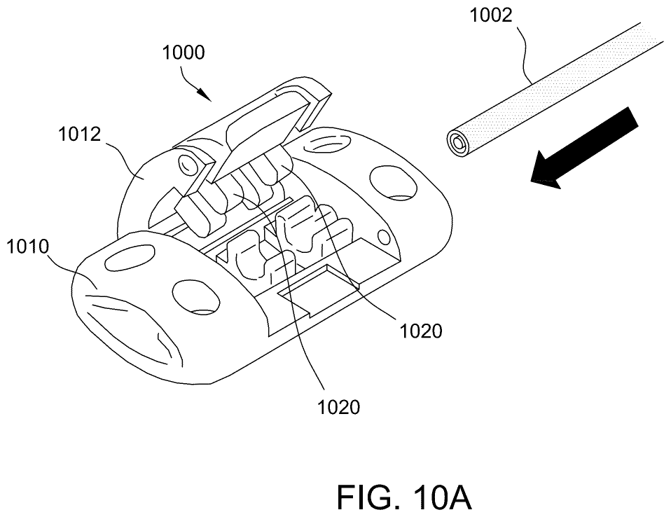

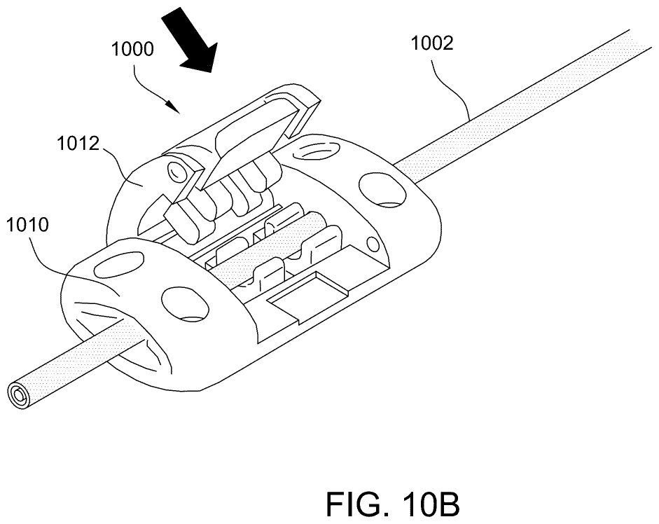

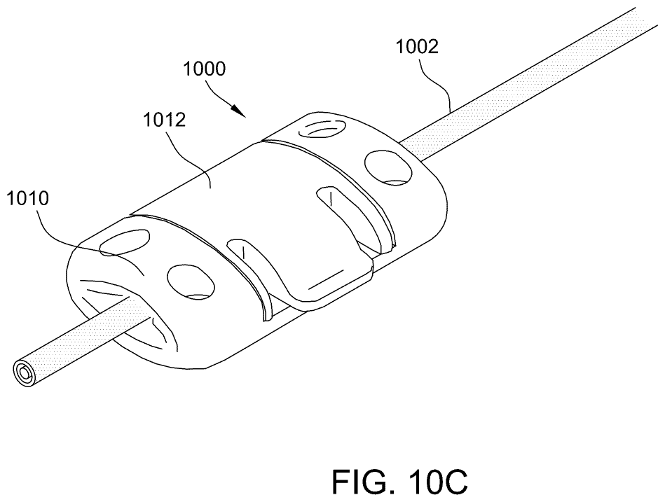

FIGS. 10A-10C are perspective views of another embodiment of an anchor assembly 1000 and lead 1002. Anchor assembly 1000 has a clamshell design, as described herein. More specifically, anchor assembly 1000 includes a body 1010 and a door 1012 pivotably coupled to the body 1010.

In FIGS. 10A and 10B, anchor assembly 1000 is shown in an unlocked position. That is, lead 1002 is able to slide relative to anchor assembly 1000. Once lead 1002 is slid through anchor assembly 1000, door 1012 may be closed onto body 1010 to transition anchor assembly 1000 to a locked position (shown in FIG. 10C). In the locked position, one or more clamps 1020 on door 1012 engage and hold lead 1002 in position, so that lead 1002 is fixedly coupled to anchor assembly 1000. To transition anchor assembly 1000 back to the unlocked position, door 1012 may be opened and disengage from lead 1002.

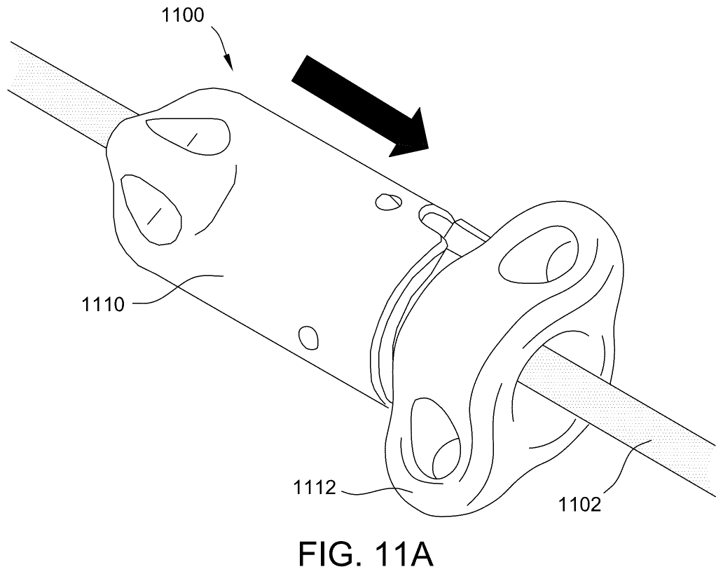

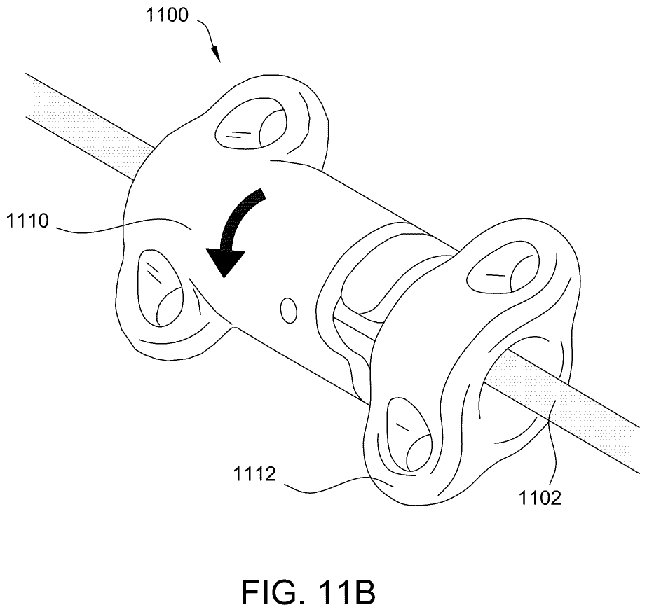

FIGS. 11A and 11B are perspective views of another embodiment of an anchor assembly 1100 and lead 1102. Anchor assembly 1100 has a twist-lock design, as described herein. More specifically, anchor assembly 1100 includes a first component 1110 rotatably coupled to a second component 1112.

In FIG. 11A, anchor assembly 1100 is shown in an unlocked position. That is, lead 1102 is able to slide relative to anchor assembly 1100. Once lead 1102 is slid through anchor assembly 1100, first component 1110 may be rotated relative to second component 1112 to transition anchor assembly 1100 to a locked position (shown in FIG. 11B). In the locked position, first component 1110 and second component 1112 component form a shear lock that secures lead 1102, so that lead 1102 is fixedly coupled to anchor assembly 1100. To transition anchor assembly 1100 back to the unlocked position, first component 1110 may be rotated back relative to second component 1112 to disengage anchor assembly 1100 from lead 1102.

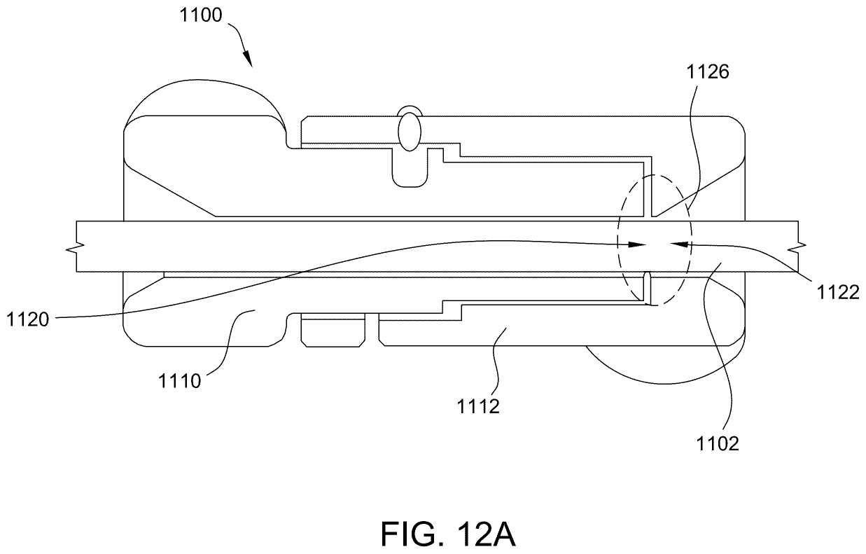

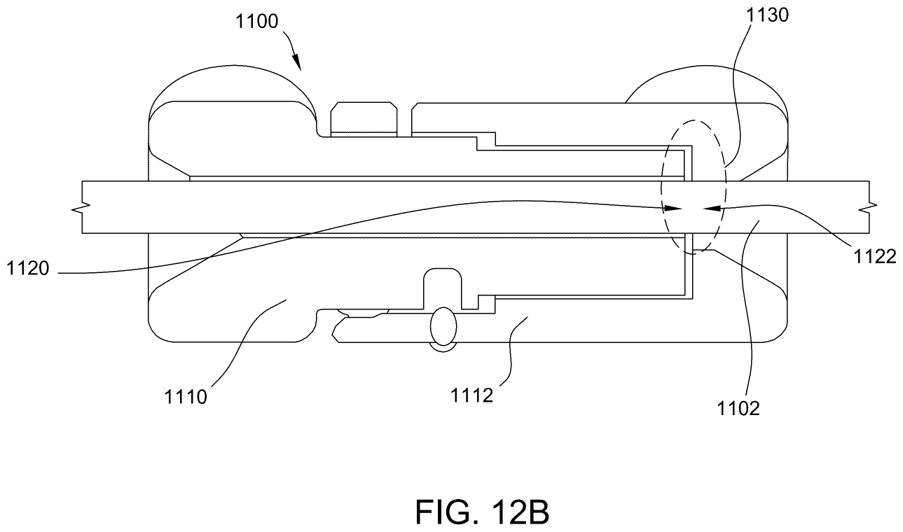

FIGS. 12A and 12B are cross-sectional views illustrating operation of anchor assembly 1100. FIG. 12A shows anchor assembly 1100 in the unlocked position, while FIG. 12B shows anchor assembly 1100 in the locked position.

In FIG. 12A, in the unlocked position, a first passageway 1120 defined through first component 1110 aligns with a second passageway 1122 defined through second component 1112 to form a clearance 1126, allowing lead 1102 to slide freely.

However, in FIG. 12B, in the locked position, after first component 1110 is rotated, first passageway 1120 is offset from second passageway 1122, forming a shear lock 1130 that secures lead 1102.

The embodiments described herein are directed to medical device anchor assemblies. An anchor assembly includes a holder defining a passageway therethrough, and a collapsible tube positioned partially within the passageway, the collapsible tube slidable relative to the holder to transition the anchor assembly between i) an unlocked position in which a medical device lead extending through the anchor assembly is slidable relative to the anchor assembly and ii) a locked position in which the medical device lead is fixedly coupled to the anchor assembly.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

1. An anchor assembly for use with a medical device lead, the anchor assembly comprising:
a holder defining a passageway therethrough; and
a collapsible tube positioned partially within the passageway, the collapsible tube slidable relative to the holder to transition the anchor assembly between i) an unlocked position in which a medical device lead extending through the anchor assembly is slidable relative to the anchor assembly and ii) a locked position in which the medical device lead is fixedly coupled to the anchor assembly.

2. The anchor assembly of claim 1, wherein the collapsible tube comprises a plurality of struts, each strut comprising at least one ramped surface that facilitates securing the medical device lead when the anchor assembly is in the locked position.

3. The anchor assembly of claim 2, wherein the at least one ramped surface includes a first portion, a second portion radially inward of the first surface, and a tapered portion extending between the first and second portions.

4. The anchor assembly of claim 1, wherein the holder comprises a plurality circumferentially arranged prongs that surround the collapsible tube.

5. The anchor assembly of claim 4, wherein at least one of the prongs defines a groove configured to engage a corresponding protrusion on the collapsible tube when the anchor assembly is in the locked position.

6. The anchor assembly of claim 1, wherein the anchor assembly comprises at least one marker band configured to indicate whether the anchor assembly is in the unlocked position or the locked position.

7. A system for use with a medical device lead, the system comprising:
an anchor assembly comprising:
a holder defining a passageway therethrough; and
a collapsible tube positioned partially within the passageway, the collapsible tube slidable relative to the holder to transition the anchor assembly between i) an unlocked position in which a medical device lead extending through the anchor assembly is slidable relative to the anchor assembly and ii) a locked position in which the medical device lead is fixedly coupled to the anchor assembly; and
a tool operable to transition the anchor assembly from the unlocked position to the locked position.

8. The system of claim 7, wherein the tool comprises:
a handle defining a slot; and
a plunger slidable within the handle, the plunger comprising an engagement end configured to push collapsible tube further into the holder while the anchor assembly is positioned in the slot.

9. The system of claim 8, wherein the tool further comprises a locking clip selectively couplable to the plunger to limit movement of the plunger relative to the handle.

10. The system of claim 8, further comprising a biasing member coupled between the handle and the plunger.

11. The system of claim 7, further comprising an unlocking tool operable to transition the anchor assembly from the locked position to the unlocked position.

12. The system of claim 11, wherein the unlocking tool comprises:
a first lever arm comprising a first engagement end configured to engage the collapsible tube; and
a second lever arm comprising a second engagement end configured to engage the holder.

13. The system of claim 7, wherein the collapsible tube comprises a plurality of struts, each strut comprising at least one ramped surface that facilitates securing the medical device lead when the anchor assembly is in the locked position.

14. The system of claim 13, wherein the at least one ramped surface includes a first portion, a second portion radially inward of the first surface, and a tapered portion extending between the first and second portions.

15. The system of claim 7, wherein the holder comprises a plurality circumferentially arranged prongs that surround the collapsible tube.

16. The system of claim 15, wherein at least one of the prongs defines a groove configured to engage a corresponding protrusion on the collapsible tube when the anchor assembly is in the locked position.

17. The system of claim 7, wherein the anchor assembly comprises at least one marker band configured to indicate whether the anchor assembly is in the unlocked position or the locked position.

18. An anchor assembly for use with a medical device lead, the anchor assembly comprising:
a body; and
a door pivotably coupled to the body, the door rotatable relative to the body to transition the anchor assembly between i) an unlocked position in which a medical device lead extending through the anchor assembly is slidable relative to the anchor assembly and ii) a locked position in which the medical device lead is fixedly coupled to the anchor assembly.

19. The anchor assembly of claim 18, wherein the door comprises at least one clamp configured to engage and hold the medical device lead when the anchor assembly is in the locked position.

20. (canceled)
